(19) United States
(12) Patent Application Publication
(10) Pub. No.: US 2017/0119287 A1
(43) Pub. Date: May 4, 2017

(54) QUANTUM-DOT SPECTROMETERS FOR USE IN BIOMEDICAL DEVICES AND METHODS OF USE

(71) Applicant: Johnson & Johnson Vision Care, Inc., Jacksonville, FL (US)

(72) Inventors: Frederick A. Flitsch, New Windsor, NY (US); Randall B. Pugh, St. Johns, FL (US)

(21) Appl. No.: 15/403,451

(22) Filed: Jan. 11, 2017

Related U.S. Application Data
(63) Continuation-in-part of application No. 14/994,390, filed on Jan. 13, 2016.
(60) Provisional application No. 62/196,513, filed on Jul. 24, 2015.

(51) Int. Cl. A61B 5/1455; A61B 5/145; A61K 49/00; A61B 5/00

(52) U.S. Cl. CPC A61B 5/1455; A61B 5/6833; A61B 5/0013; A61B 5/6831; A61B 5/6843; A61B 5/14532; A61B 5/4839; A61K 49/0004; A61B 2562/0285; A61B 2562/12

(57) ABSTRACT

Device and methods for the incorporation of Quantum-Dots for spectroscopic analysis into biomedical devices are described. In some examples, the Quantum-Dots act as light emitters, light filters or analyte specific dyes. In some examples, a field of use for the apparatus and methods may include any biomedical device or product that benefits from spectroscopic analysis.

### CROSS REFERENCE TO RELATED APPLICATIONS

This application is a continuation in part of U.S. patent application Ser. No. 14/994,390 filed on Jan. 13, 2016, which claims the benefit of U.S. Provisional Application No. 62/196,513 filed Jul. 24, 2015.

### BACKGROUND OF THE INVENTION

1. Field of the Invention

Quantum-dot spectrometers for use in biomedical devices are described herein. In some exemplary embodiments, the devices' functionality involves collecting biometric information to perform personalized bioanalysis for the user of the device.

2. Discussion of the Related Art

Recently, the number of medical devices and their functionality has begun to rapidly develop. These medical devices may include, for example, implantable pacemakers, electronic pills for monitoring and/or testing a biological function, surgical devices with active components, contact lenses, infusion pumps, and neurostimulators. These devices are often exposed to and interact with biological and chemical systems making the devices optimal tools for collecting, storing, and distributing biometric data.

Some medical devices may include components such as semiconductor devices that perform a variety of functions including biometric collection, and may be incorporated into many biocompatible and/or implantable devices. Such semiconductor components require energy and, thus, energization elements must also be included in such biocompatible devices. The addition of self-contained energy in a biomedical device capable of collecting biometric information would enable the device to perform personalized biometric analysis for the user of the device.

One aspect of biometric information collection has focused on the ability to pair an analyte to a corresponding enzyme such as glucose to glucose oxidase for the detection of glucose in a fluid medium. Another aspect of biometric information collection may focus on the use of light where a light source shines light through a medium which is in turn collected by a detector and analyzed for the amount of light absorbed, similar to a spectrometer. Spectrometers are widely used in physical, chemical, and biological research; however, current micro-spectrometer designs mostly use interference filters and interferometric optics that limit their photon efficiency, resolution, and spectral range. Nevertheless, the miniaturization possible with the development of techniques and reagents that utilize quantum-dots in supporting the acquisition of spectroscopic data may allow for significant advances in the ability of biomedical devices to sense chemical states of their environments.

A quantum-dot (QD) is a nanocrystal commonly made of semiconductor materials. When crystals are “nano-sized” they become small enough to exhibit quantum mechanical properties. Technologies around QDs exploit this quantum mechanical behavior to result in interesting optical properties for the QDs. Therefore, novel devices for biomedical purposes for the use of quantum-dots and for quantum-dot spectrometry may be useful.

### SUMMARY OF THE INVENTION

Accordingly, devices and methods for the use of QDs as emission sources, filters, dyes and as narrow and broadband spectrometers on or in powered biomedical devices may enable the powered biomedical devices to specifically and accurately detect analytes on or in the body of a user. In some examples, the use of QDs may be used in a biomedical device which operates in a non-invasive manner and irradiates through the skin of the user.

Quantum-dots are extremely small entities that can be manufactured with high levels of consistency and purity. Since the quantum-dot manufacturing process may be tuned to different sizes and materials, a nearly arbitrary amount of frequencies may be tuned for the spectral response from a type of QD. As emission sources, therefore, fine line fluorescence sources may be formed from the excitation of QDs with their resultant high yield fluorescence emission. For the use of QDs as filters, a tunable transmission response may be obtained. Therefore, it may be easy to create spectrometers comprising hundreds of unique and tuned spectral filters to create the perspective of broadband spectroscopy. Still further spectral relevance of QDs may arise from the fact that individual QDs may have molecules that bind to the surface and quench their fluorescence. These quenching molecules may be selected and designed to bind to analytes and in so doing decouple from the QD that they are quenching, resulting in sensitive fluorescence probes for analyte study.

One general aspect of the present invention includes forming a biomedical device including an energization element including a first and second current collector, a cathode an anode and an electrolyte. The biomedical device may also comprise a quantum-dot spectrometer which may include a quantum-dot light emitter, a photodetector, and a means of communicating information from the quantum-dot spectrometer to a user. The quantum-dot spectrometer is powered by the energization element. The biomedical device may also include a bandage device. This bandage device may contain the energization element and the quantum-dot spectrometer. The bandage device attaches the quantum-dot spectrometer to the skin of the user, in some examples with an adhesive. In other examples, the biomedical device may comprise clips, cuffs, straps and other attachment devices other than a bandage. In some of these examples, the biomedical device may operate as a non-invasive device. The biomedical device may comprise multiple detector regions which may be deployed at different lengths from the light emitter. In some examples, a constant distance between the detector and the light source may be formed by shaping the detector in a circular pattern to form a circular device. In some examples both the first and second detectors may be circularly shaped with different radii. Quantum dots with detector elements may be located along the bodies of the first and second detectors, thus as photons emanate from the light sources they will take paths through the skin in all the directions which may intersect the circular detectors while proceeding through the same length of skin tissue. In some examples, the first detector body may a smaller distance than the second detector and may have interruptions in its body to allow light to simultaneously proceed to both the first and second detector. In some examples there may be more than two detectors. The biomedical device may include electronic circuits of various kinds to control power, perform algorithms for calculation of absorbance aspects, and to provide communication aspects to the device. The wireless communication device may be used to ultimately pass data, perhaps through various communications layers such as routers, switches and the like, onto a server. The server may comprise means of performing intensive algorithmic calculations on the data communicated which may be used to extract information about particular analytes. In some examples, a server may receive data wherein the server may have capabilities to perform cognitive algorithms upon the data.

Implementations may include a method of analyzing analytes. The method may include fabricating a quantum-dot photodetector into a biomedical device. As well, a photon emitter may be included into the biomedical device. The method may include connecting the photon emitter and photodetector to an integrated circuit controller where this controller may be capable of directing the functionality of the quantum-dot emitter and photodetector. The method may further include emitting a wavelength band from the photon emitter. The method may include receiving transmitted photons into the quantum dot photodetector. In some implements the method may continue with analyzing the absorbance of an analyte based on the intensity of photons received. The biomedical device may comprise an energization element which may include a first and second current collector, a cathode, and anode and an electrolyte where the spectrometer is powered by the energization element.

One general aspect of the present invention includes a biomedical device comprising an energization element. The biomedical device may include an external encapsulation boundary. The external encapsulation boundary may include a reentrant cavity which creates an external region that may be generally surrounded by the biomedical device while allowing fluid to flow in and out from the environment of the biomedical device. The encapsulation layers of the biomedical device may allow light to pass through them in important spectral bands. The reentrant channel may be lined by photon emitters and detectors.

Implementations may include a method of analyzing analytes including obtaining a biomedical device comprising a quantum dot, wherein the biomedical device itself comprises a photon source and a first photodetector which provide an ability to probe for spectral data through skin of a user and pass light through the skin of the user. The distance between this first photodetector and the photon source may be a first distance. A second photodetector may be located on the skin where this second photodetector is at a different distance from the light source than the first photodetector. In this way there are at least two different lengths of skin through which the light proceeds through. The device with both the first photodetector and the second photodetector and a light source may be located in contact with the user's skin. The device may be used to measure absorption through the user's skin using both photodetectors. The resulting data collected by the measurement may be communicated to an external receiver. The first detector may comprise quantum dots.

Implementations may include methods of monitoring a patient's glucose level. The methods may include associating a data level in a controller, wherein the data level corresponds to acceptable detection measurements obtained with a biomedical device comprising a quantum dot, wherein the detection measurements correlate to a concentration of the glucose. As well, the methods may include placing the biomedical device comprising a quantum dot in contact with the patient's skin, wherein the biomedical device comprises a photon source and at least a first photodetector which provide an ability to probe for spectral data through skin of the patient. Furthermore, the methods may include monitoring the detection measurements which correlate to the concentration of the glucose in the patient's body, wherein the detection measurement are obtained in a non-invasive means through the skin of the patient. The implementation may include communicating the detection measurements to at least one of the patient and a medical practitioner of the patient. As well, the method may include identifying a pattern in the detection measurements that are tied to the patient. As well, the pattern may result in adjusting the data levels in the controller based on the observed pattern.

In some implementations of the present invention, the biomedical device may include a quantum-dot light emitter installed to emit light through one side of the sidewall of the cavity through the intervening space of the cavity. The light may further proceed through the opposite or distal side of the sidewall of the cavity. On the other side may be numerous photodetectors installed within the external encapsulation boundary. The biomedical device may also include a radio frequency transceiver and an analog-to-digital converter. A signal from the photodetector may be converted by the analog-to-digital converter into a data value that may be transmitted by the radio frequency transceiver. In some examples the biomedical device may be a contact lens or an electronic pill. In examples of an electronic pill, the pill may also comprise a release mechanism controllable to release medicament. The detector may form a feedback loop for the device and therefore may adjust the amount of medicament dispersed by the pill.

In some examples of a biomedical device comprising a QD spectrometer, the signal received at the photodetector may be converted to a digital signal and communicated to an external receiver. This external receiver may include a processor that may execute an algorithm which calculates a concentration of an analyte and then determines the concomitant release in medicament that is desired. The external receiver may transmit data and control signals to the biomedical device.

Implementations of the present invention may include a biomedical device including an energization element. The biomedical device may also include an external encapsulation boundary wherein at least a portion of the boundary comprises an electrically controlled pore. The pore may be operative to allow a fluid sample to pass into the biomedical device from an external region. The biomedical device may also include a microfluidic processing chip which may mix fluid samples and reagents. Reagents in the microfluidic processing chip may include analyte specific dyes. The biomedical device may include a quantum-dot light emitter which may emit light through a portion of the microfluidic processing chip. The device may also include a photodetector installed on a distal position of the microfluidic processing chip. The device may also include a radio frequency transceiver. The implement may also include an analog to digital converter where a signal from the photodetector may be converted to a digital data value that is transmitted outside the biomedical device by the radio frequency transceiver. These examples may include biomedical devices which are contact lenses, electronic pills and electronic pills capable to release medicament based on the signal received at the photodetector.

One general aspect includes a biomedical device as an electronic pill, where the electronic pill includes a release mechanism controllable to release a quantum-dot dye into the cavity, where the dye reacts with analyte molecules and allows the quantum light emitter to excite the quantum-dot dye to emit light. The biomedical device also includes an energization element; an external encapsulation boundary, where at least a portion of the boundary includes an electrically controlled pore operative to allow a fluid sample to pass into the biomedical device from an external region; a microfluidic processing chip operative to mix the fluid sample with a reagent including an analyte specific dye; a quantum-dot light emitter installed to emit light through a portion of the microfluidic processing chip; a photodetector installed on a distal side of the microfluidic processing chip from the quantum-dot light emitter, where light emitted by the quantum-dot light emitter proceeds through a top surface of the microfluidic processing chip, through a sample analysis region of the microfluidic processing chip, through a bottom surface of the microfluidic processing chip and into the photodetector; a radio frequency transceiver; and an analog-to-digital converter, where a signal from the photodetector is converted to a digital data value that is transmitted. The electronic pill may control its release of medicament based on the captured data. The release of medicament may be adjusted by a controller which acts in response to receipt of converted digital data value.

In some examples the biomedical device may comprise a portion that is controllable to release a quantum-dot dye into the microfluidic processing chip. The dye may react with analyte molecules and the reaction may allow the quantum-dot light emitter to excite the quantum-dots without the presence of quenching molecules which may extinguish the characteristic emission from the quantum-dots.

### BRIEF DESCRIPTION OF THE DRAWINGS

The foregoing and other features and advantages of the invention will be apparent from the following, more particular description of preferred embodiments of the invention, as illustrated in the accompanying drawings:

FIG. 1 illustrates a non-invasive spectral analysis through a user's skin.

FIG. 2 illustrates how a spectral band may be analyzed with quantum-dot based filters.

FIG. 3 illustrates a processor that may be used to implement some embodiments of the present invention.

FIG. 4 illustrates an exemplary functional structure model for a biomedical device with a quantum-dot spectrometer.

FIGS. 5 A-C illustrate an exemplary Quantum-Dot Spectrometer in a biomedical device.

FIG. 6 illustrates an exemplary quantum-dot based fluorescence dye.

FIG. 7 illustrates an exemplary flow diagram for sample analyte detection by quantum-dot based spectroscopy.

FIG. 8 illustrates exemplary method steps that may be used to monitor analyte levels of a user wearing the non-invasive quantum dot device according to aspects of the present invention.

FIG. 9 illustrates exemplary method steps that may be used to treat the glucose levels of a user wearing the non-invasive quantum dot device according to aspects of the present invention.

FIG. 10 illustrates an exemplary detector and light source which may be included in a quantum dot analysis device which non-invasively probes the skin layers of a user.

FIG. 11 illustrates an exemplary bandage device incorporating quantum dot based elements which may be used for glucose analysis.

### DETAILED DESCRIPTION OF THE INVENTION

Spectroscopy utilizing quantum-dots as emission sources, filters and dyes which may be used in biomedical devices are disclosed in this application. In the following sections, detailed descriptions of various examples are described. The descriptions are exemplary embodiments only, and various modifications and alterations may be apparent to those skilled in the art. Therefore, the examples do not limit the scope of this application. Quantum-dot based spectrometers for use in biomedical devices, and the structures that contain them, may be designed for use in devices such as non-invasive quantum dot devices and electronic pills. In some examples, spectroscopy methods utilizing quantum-dots for use in biomedical devices may be designed for use in, or proximate to, the body of a living organism.

### Glossary

In the description and claims below, various terms may be used for which the following definitions will apply:

“Anode” as used herein refers to an electrode through which electric current flows into a polarized electrical device. The direction of electric current is typically opposite to the direction of electron flow. In other words, the electrons flow from the anode into, for example, an electrical circuit.

“Binder” as used herein refers to a polymer that is capable of exhibiting elastic responses to mechanical deformations and that is chemically compatible with other energization element components. For example, binders may include electroactive materials, electrolytes, polymers, and the like.

“Biocompatible” as used herein refers to a material or device that performs with an appropriate host response in a specific application. For example, a biocompatible device does not have toxic or injurious effects on biological systems.

“Cathode” as used herein refers to an electrode through which electric current flows out of a polarized electrical device. The direction of electric current is typically opposite to the direction of electron flow. Therefore, the electrons flow into the cathode of the polarized electrical device, and out of, for example, the connected electrical circuit.

“Coating” as used herein refers to a deposit of material in thin forms. In some uses, the term will refer to a thin deposit that substantially covers the surface of a substrate it is formed upon. In other more specialized uses, the term may be used to describe small thin deposits in smaller regions of the surface.

“Electrode” as used herein may refer to an active mass in the energy source. For example, it may include one or both of the anode and cathode.

“Energized” as used herein refers to the state of being able to supply electrical current or to have electrical energy stored within.

“Energy” as used herein refers to the capacity of a physical system to do work. Many uses of the energization elements may relate to the capacity of being able to perform electrical actions.

“Energy Source” or “Energization Element” or “Energization Device” as used herein refers to any device or layer which is capable of supplying energy or placing a logical or electrical device in an energized state. The energization elements may include batteries. The batteries may be formed from alkaline type cell chemistry and may be solid-state batteries or wet cell batteries.

“Fillers” as used herein refer to one or more energization element separators that do not react with either acid or alkaline electrolytes. Generally, fillers may include substantially water insoluble materials such as carbon black; coal dust; graphite; metal oxides and hydroxides such as those of silicon, aluminum, calcium, magnesium, barium, titanium, iron, zinc, and tin; metal carbonates such as those of calcium and magnesium; minerals such as mica, montmorollonite, kaolinite, attapulgite, and talc; synthetic and natural zeolites such as Portland cement; precipitated metal silicates such as calcium silicate; hollow or solid polymer or glass microspheres, flakes and fibers; and the like.

“Functionalized” as used herein refers to making a layer or device able to perform a function including, for example, energization, activation, and/or control.

“Ionizing Salt” as used herein refers to an ionic solid that will dissolve in a solvent to produce dissolved ions in solution. In numerous examples, the solvent may comprise water. “Mold” as used herein refers to a rigid or semi-rigid object that may be used to form three-dimensional objects from uncured formulations. Some exemplary molds include two mold parts that, when opposed to one another, define the structure of a three-dimensional object.

“Power” as used herein refers to work done or energy transferred per unit of time.

“Rechargeable” or “Re-energizable” as used herein refer to a capability of being restored to a state with higher capacity to do work. Many uses may relate to the capability of being restored with the ability to flow electrical current at a certain rate for certain, reestablished time periods.

“Reenergize” or “Recharge” as used herein refer to restoring to a state with higher capacity to do work. Many uses may relate to restoring a device to the capability to flow electrical current at a certain rate for a certain reestablished time period.

“Released” as used herein and sometimes referred to as “released from a mold” means that a three-dimensional object is either completely separated from the mold, or is only loosely attached to the mold, so that it may be removed with mild agitation.

“Stacked” as used herein means to place at least two component layers in proximity to each other such that at least a portion of one surface of one of the layers contacts a first surface of a second layer. In some examples, a coating, whether for adhesion or other functions, may reside between the two layers that are in contact with each other through said coating.

“Traces” as used herein refer to energization element components capable of connecting together the circuit components. For example, circuit traces may include copper or gold when the substrate is a printed circuit board and may typically be copper, gold or printed film in a flexible circuit. A special type of “Trace” is the current collector. Current collectors are traces with electrochemical compatibility that make the current collectors suitable for use in conducting electrons to and from an anode or cathode in the presence of electrolyte.

Recent developments in biomedical devices including, for example, non-invasive quantum dot devices, have enabled functionalized biomedical devices that may be energized. The energized biomedical devices may comprise the necessary elements to collect spectra and analyze the concentration and qualitative presence of analytes of users using embedded micro-electronics. Additional functionality using micro-electronics may include, for example, audio, visual, and haptic feedback to the user. In some embodiments, the quantum-dot spectrometers for use in biomedical devices may be in wireless communication with one or more wireless device(s) and receive signal data that may be used in real time for the determination of an abnormal analyte concentration and correlated cause. The wireless device(s) may include, for example, a smart phone device, a tablet, a personal computer, a FOB, an MP3 player, a PDA, and other similar devices.

### Energized Non-invasive Quantum Dot Device

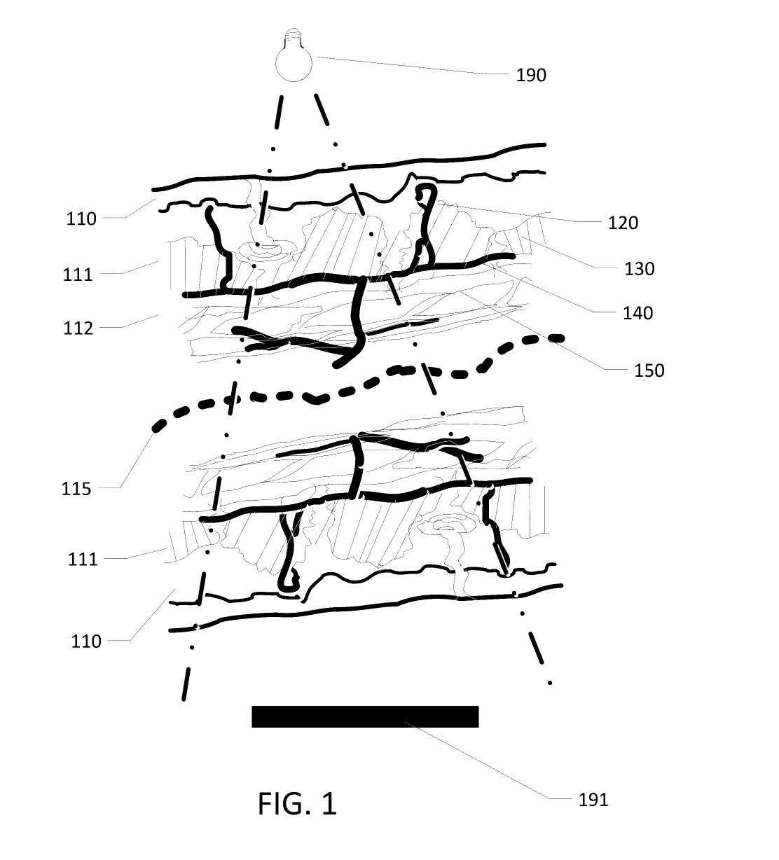

Referring to FIG. 1, an illustration of a quantum dot based non-invasive monitoring device is provided with exemplary illustration of skin layers depicted. In an example, the skin layers may represent the skin flap between the thumb and forefinger. Or, the skin layers may represent the skin of the ear lobe or ear body. In some other examples, a device may pinch a skin flap for analysis. In still further examples, as will be depicted in following sections the path of light illustrated may be created by depressing the light source and the detector devices into flexible portions of the skin of a user. Referring again to the illustration, the epidermal layer 110 is illustrated on either side of the skin layers. The dermis layer 111 lies beneath the epidermal layer 110. There may be fluids and biomolecules or other analytes of interest that may be found in the top layers of the skin. As well, capillaries 120 may be found in this region. The subdermal layer 112 may have significant vascular structure 140 and fat tissue 130 as well as intra-tissue regions 150 that may be filled with fluid including various analytes. A light source 190 may be used to irradiate the skin layers. There may be numerous spectral regions of interest that may be irradiated by the light source. Depending on the thickness of the skin layers being probed, the spectral regions may include in non-limiting examples, infrared, near infrared, ultra-violet, and visible regions. The light is illustrated as proceeding through the skin layers and then through intervening layers 115 depending on the location of the skin. The dotted line depicts these layers 115 between the skin surfaces is intended to illustrate a variable amount of tissue between the upper skin layers through which the light may travel. In some examples, the light source 190 may project in numerous directions and each of these may include a different type of quantum dot element as will be described in following paragraphs. In other examples, the light source 190 may be a broad spectrum light source that traverses the skin layers to a detector element 191. Broad spectrum light sources may emit light from the UV to the infrared which in some examples may be a band of wavelengths between 100 nm to 15 microns. In some examples, the light source may comprise an LED infrared light source. The infrared led light source may be made in small form factors and may comprise a relatively narrow band around a central target frequency. Light emitting diodes may be chosen to emit at visible and ultraviolet wavelengths as well. In some other examples, the light source may comprise a light emitting laser device. Infrared lasers may emit coherent, intense and collimated light over a very narrow wavelength regime. The detector element 191 may include various quantum dot filters above a collection of discrete photosensitive detectors as discussed in greater detail subsequently. A number of detector elements may allow for the simultaneous acquisition of different regions of the light spectra.

### Quantum-Dot Spectroscopy

Small spectroscopy devices may be of significant aid in creating biomedical devices with the capability of measuring and controlling concentrations of various analytes for a user. For example, the metrology of glucose may be used to control variations of the material in patients and after treatments with medicines of various kinds. Current microspectrometer designs mostly use interference filters and interferometric optics to measure spectral responses of mixtures that contain materials that absorb light. In some examples a spectrometer may be formed by creating an array composed of quantum-dots. A spectrometer based on quantum-dot arrays may measure a light spectrum based on the wavelength multiplexing principle. The wavelength multiplexing principle may be accomplished when multiple spectral bands are encoded and detected simultaneously with one filter element and one detector element, respectively. The array format may allow the process to be efficiently repeated many times using different filters with different encoding so that sufficient information is obtained to enable computational reconstruction of the target spectrum. An example may be illustrated by considering an array of light detectors such as that found in a CCD camera. The array of light sensitive devices may be useful to quantify the amount of light reaching each particular detector element in the CCD array. In a broadband spectrometer, a plurality, sometimes hundreds, of quantum-dot based filter elements are deployed such that each filter allows light to pass from certain spectral regions to one or a few CCD elements. An array of hundreds of such filters laid out such that an illumination light passed through a sample may proceed through the array of QD filters and on to a respective set of CCD elements for the QD filters. The simultaneous collection of spectrally encoded data may allow for a rapid analysis of a sample.

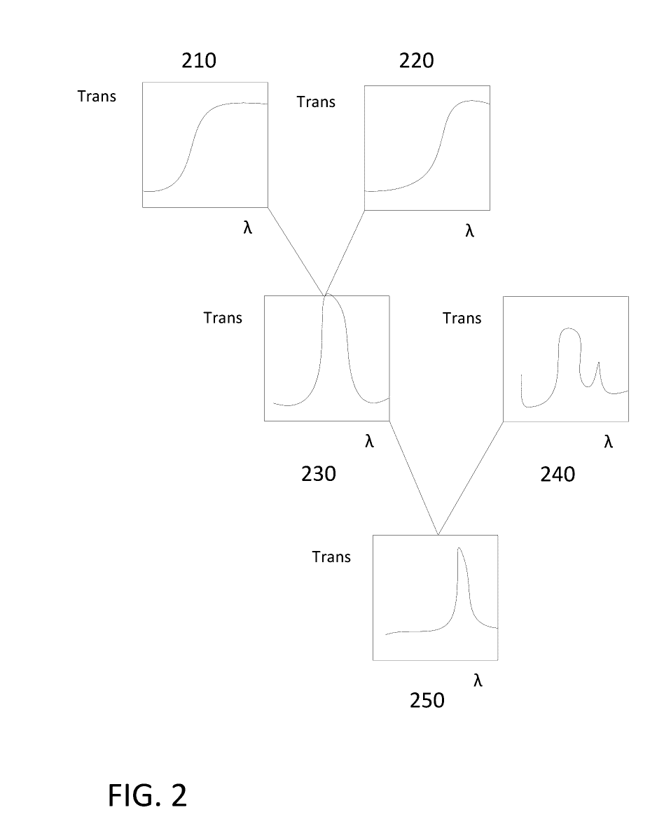

Narrow band spectral analysis examples may be formed by using a smaller number of QD filters surrounding a narrow band. In FIG. 2 an illustration of how a spectral band may be observed by a combination of two filters is illustrated. It may also be clear that the array of hundreds of filters may be envisioned as a similar concept to that in FIG. 2 repeated may times.

If FIG. 2, a first QD filter 210 may have an associated spectral transmission response as illustrated and indicated as Trans. A second QD filter 220 may have a shifted associated spectral transmission associated with a different nature of the quantum-dots included in the filter, for example, the QDs may have a larger diameter in the QD filter of 220. The difference curve of a flat irradiance of light of all wavelength (white light) may result from the difference of the absorption result from light that traverses second QD filter 220 and that traverses first QD filter 210. Thus, the effect of irradiating through these two filters is that the difference curve would indicate spectral response in the band 230 depicted. When an analyte is introduced into the light path of the spectrometer, where the analyte has an absorption band in the UV/Visible spectrum, and possible in the infrared, the result would be to modify the transmission of light in that spectral band as shown by spectrum 240. The difference from 230 to 240 results in a transmission spectrum 250 for the analyte in the region defined by the two quantum-dot filters. Therefore, a narrow spectral response may be obtained by a small number of filters. In some examples, redundant coverage by different filter types of the same spectral region may be employed to improve the signal to noise characteristics of the spectral result.

The absorption filters based on QDs may include QDs that have quenching molecules on their surfaces. These molecules may stop the QD from emitting light after it absorbs energy in appropriate frequency ranges. More generally, the QD filters may be formed from nanocrystals with radii smaller than the bulk excitation Bohr radius, which leads to quantum confinement of electronic charges. The size of the crystal is related to the constrained energy states of the nanocrystal and generally decreasing the crystal size has the effect of a stronger confinement. This stronger confinement affects the electronic states in the quantum-dot and results in an increase in the effective bandgap, which results in shifting to the blue wavelengths both of optical absorption and fluorescent emission. There have been many spectral limited sources defined for a wide array of quantum-dots that may be available for purchase or fabrication and may be incorporated into biomedical devices to act as filters. By deploying slightly modified QDs such as by changing the QD's size, shape and composition it may be possible to tune absorption spectra continuously and finely over wavelengths ranging from deep ultraviolet to mid-infrared. QDs may also be printed into very fine patterns.

### Diagrams for Electrical and Computing System

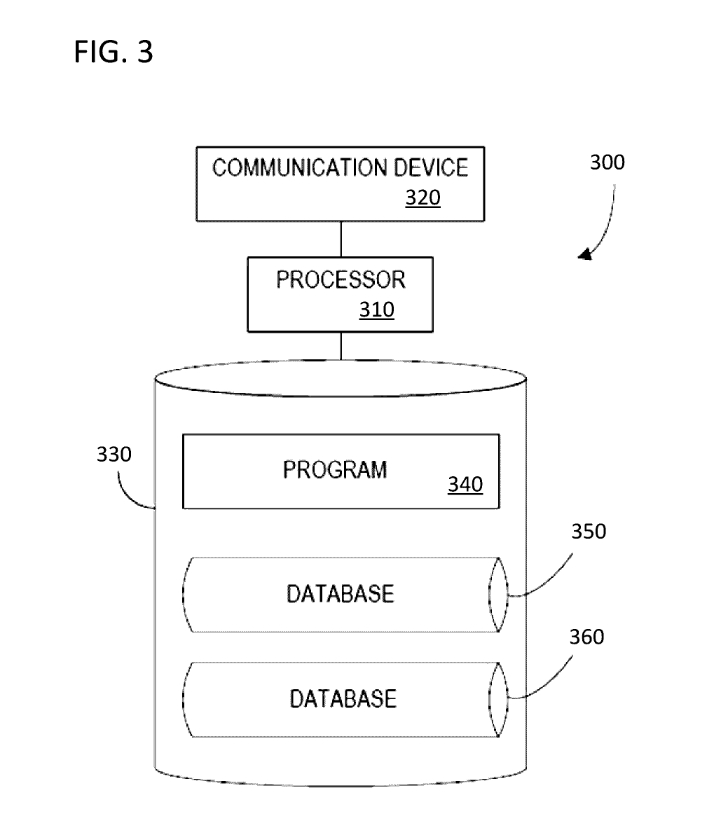

Referring now to FIG. 3, a schematic diagram of a processor that may be used to implement some aspects of the present disclosure is illustrated. The controller 300 may include one or more processors 310, which may include one or more processor components coupled to a communication device 320. In some embodiments, a controller 300 may be used to transmit energy to the energy source placed in the device.

The processors 310 may be coupled to the communication device 320 configured to communicate energy via a communication channel. The communication device 320 may be used to electronically communicate with components within the media insert, for example. The communication device 320 may also be used to communicate, for example, with one or more controller apparatus or programming/interface device components

The processor 310 is also in communication with a storage device 330. The storage device 330 may comprise any appropriate information storage device, including combinations of magnetic storage devices, optical storage devices, and/or semiconductor memory devices such as Random Access Memory (RAM) devices and Read Only Memory (ROM) devices.

The storage device 330 may store a software program 340 for controlling the processor 310. The processor 310 performs instructions of a software program 340, and thereby operates in accordance with the present invention. For example, the processor 310 may receive information descriptive of media insert placement, active target zones of the device. The storage device 330 may also store other pre-determined biometric related data in one or more databases 350 and 360. The database may include, for example, predetermined retinal zones exhibiting changes according to cardiac rhythm or an abnormal condition correlated with the retinal vascularization, standard measurement thresholds, metrology data, and specific control sequences for the system, flow of energy to and from a media insert, communication protocols, and the like. The database may also include parameters and controlling algorithms for the control of the biometric based monitoring system that may reside in the device as well as data and/or feedback that can result from their action. In some embodiments, that data may be ultimately communicated to/from an external reception wireless device.

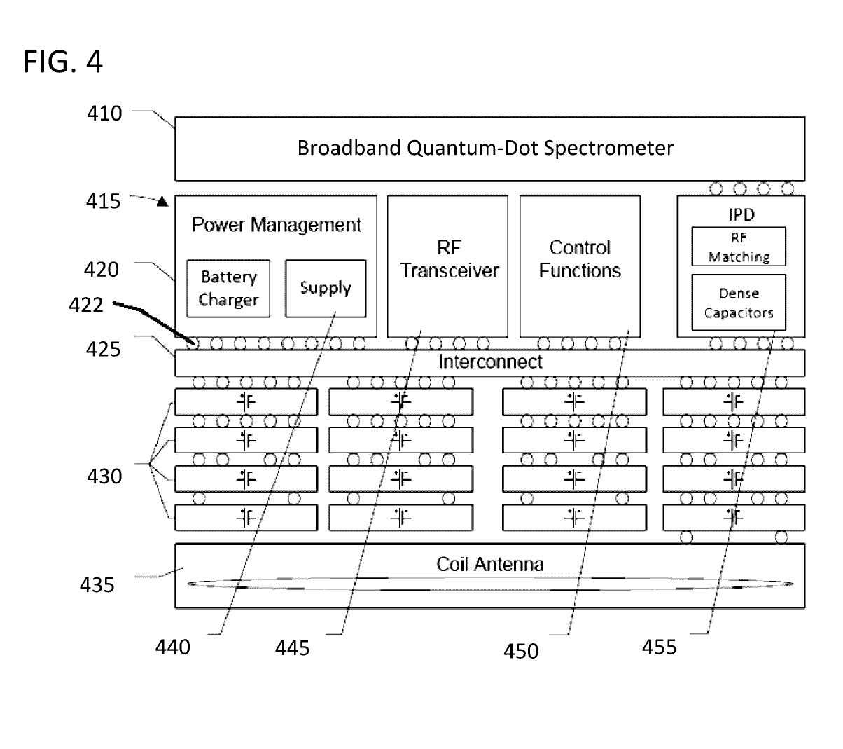

In some embodiments according to aspects of the present invention, a single and/or multiple discrete electronic devices may be included as discrete chips. In other embodiments, energized electronic elements may be included in a media insert in the form of stacked integrated components. Referring now to FIG. 4, a schematic diagram of an exemplary cross section of stacked die integrated components implementing a quantum-dot spectrometer system 410 is depicted. The quantum-dot spectrometer may be, for example, a glucose monitor, a retinal vascularization monitor, a visual scanning monitor, or any other type of system useful for providing spectrophometric information about the user. In particular, a media insert may include numerous layers of different types which are encapsulated into contours consistent with the environment that they will occupy. In some embodiments, these media inserts with stacked integrated component layers may assume the entire shape of the media insert. Alternatively, in some cases, the media insert may occupy just a portion of the volume within the entire shape.

In many examples the concept of a media insert has been invoked. Numerous examples may be found of a media insert being an entity that is encapsulated within the body of an advanced contact lens. However, in some examples, a media insert may also refer to a similar entity that may contain numerous components including such elements as energization elements, electronics circuits, integrated circuits, sensors, processors and the like. The media insert may be formed into this self-contained device which may be inserted into the body of other generic devices to give them functionality. In a non-limiting example of a device type which could incorporate other types of media inserts, a bandage device may include a media insert comprising a quantum dot spectrometer system which is contained within the body of a plastic film coated with adhesive to attach the device to a user's skin. Other forms of devices may comprise a generic media insert for functionality as described herein.

As shown in FIG. 4, there may be batteries 430 used to provide energization. In some embodiments, these batteries 430 may comprise one or more of the layers that may be stacked upon each other with multiple components in the layers and interconnections there between. The batteries 430 are depicted as thin film batteries for exemplary purposes; however, there may be numerous other energization elements consistent with the embodiments herein, including operation in both stacked and non-stacked embodiments. As a non-limiting alternative example, cavity based laminate form batteries with multiple cavities may perform equivalently or similarly to the depicted thin film batteries.

In some embodiments, there may be additional interconnections between two layers that are stacked upon each other. In the state of the art there may be numerous manners to make these interconnections; however, as demonstrated the interconnection may be made through solder ball interconnections 422 between the layers. In some embodiments only these connections may be required; however, in other cases other solder balls may contact other interconnection elements, as for example with a component having through layer vias such as might be present in an integrated passive device 455.

In other layers of the stacked integrated component media insert, an interconnect layer 425 may be dedicated for the interconnections of two or more of the various components in the interconnect layers. The interconnect layer 425 may contain, vias and routing lines that can pass signals from various components to others. For example, interconnect layer 425 may provide the various battery elements connections to a power management unit 420 that may be present in a technology layer 415. The power management unit 420 may have circuitry dedicated to supplying voltage sources with controlled characteristics 440. Other components in the technology layer 415 may include, for example, a transceiver 445, control components 450 and the like. In addition, the interconnect layer 425 may function to make connections between components in the technology layer 415 as well as components outside the technology layer 415; as may exist for example in the integrated passive device 455. There may be numerous manners for routing of electrical signals that may be supported by the presence of dedicated interconnect layers such as interconnect layer 425.

In some embodiments, the technology layer 415, like other layer components, may be included as multiple layers as these features represent a diversity of technology options that may be included in media inserts. In some embodiments, one of the layers may include CMOS, BiCMOS, Bipolar, or memory based technologies whereas the other layer may include a different technology. Alternatively, the two layers may represent different technology families within a same overall family; as for example one layer may include electronic elements produced using a 0.5 micron CMOS technology and another layer may include elements produced using a 20 nanometer CMOS technology. It may be apparent that many other combinations of various electronic technology types would be consistent within the art described herein.

In some embodiments, the media insert may include locations for electrical interconnections to components outside the insert. In other examples; however, the media insert may also include an interconnection to external components in a wireless manner. In such cases, the use of antennas in an antenna layer 435 may provide exemplary manners of wireless communication. In many cases, such an antenna layer 435 may be located, for example, on the top or bottom of the stacked integrated component device within the media insert.

In some of the embodiments discussed herein, the energization elements such as batteries 430 may be included as elements in at least one of the stacked layers themselves. It may be noted as well that other embodiments may be possible where the batteries 430 are located externally to the stacked integrated component layers. Still further diversity in embodiments may derive from the fact that a separate battery or other energization component may also exist within the media insert, or alternatively these separate energization components may also be located externally to the media insert. In these examples, the functionality may be depicted for inclusion of stacked integrated components, it may be clear that the functional elements may also be incorporated into biomedical devices in such a manner that does not involve stacked components and still be able to perform functions related to the embodiments herein.

Components of the quantum-dot spectrometer system 410 may also be included in a stacked integrated component architecture. In some embodiments, the quantum-dot spectrometer system 410 components may be attached as a portion of a layer. In other embodiments, the entire quantum-dot spectrometer system 410 may also comprise a similarly shaped component as the other stacked integrated components. In some alternative examples, the components may not be stacked but laid out in the peripheral regions of the non-invasive quantum dot device or other biomedical device, where the general functional interplay of the components may function equivalently however the routing of signals and power through the entire circuit may differ.

When constructing a quantum-dot spectrometer system 410 in a biomedical device, size may be an integral factor. Quantum-dot emitters may be fashioned in a manner similar to the formation of light emitting diodes. Layers of materials may surround the quantum-dots to create light emitting diodes with the quantum-dots. Organic layers may act as electron donors and as hole donors into the quantum-dot layer. In a non-limiting example, the QDs may be sandwiched between electron transport layers and hole transport layers. Application of electric potential to electrodes connected to the electron transport layer and the hold transport layer excite the QD into photoluminescence at a wavelength band characteristic of the QDs. Examples of electron transport layers and hole transport layers may include tris-(8-hydroxyquinoline) aluminum; bathocuproine; 4,4′-N,N′-dicarbazolylbiphenyl; poly(2-(6-cyano-6′-methylheptyloxy)-1,4-phenylene); poly[(9,9-dihexylfluorenyl-2,7-diyl)-co-(1,4-{benzo-[2,1′,3]thiadiazole})]; poly[2-methoxy-5-(2-ethylhexyloxy)-1,4-phenylenevinylene]; 4,4-bis[N-(1-naphyl)-N-phenylamino] biphenyl; 2-(4-biphenylyl)-5-(4-tertbutylphenyl)-1,3,4 oxadiazole; poly-3,4-ethylene dioxythiophene; poly(9,9′-dioctylfluorene-co-N-(4-butylphenyl)diphenylamine; perfluoro-cyclobutane; poly(phenylene vinylene); 3-(4-Biphenylyl)-4-phenyl-5-tertbutylphenyl-1,2,4-triazole; Poly[(9,9-dioctylfluorenyl-2,70diyl)-co-(4-4′-(N-(4-secbutylphenyl)) diphenylamine)]; 1,3,5-tris(N-phenylbenzimidazole-2-yl)-benzene; and N,N′-diphenyl-N,N′-bis(3-methylphenyl)-(1,1′-biphenyl)-4,4′-diamine as non-limiting examples.

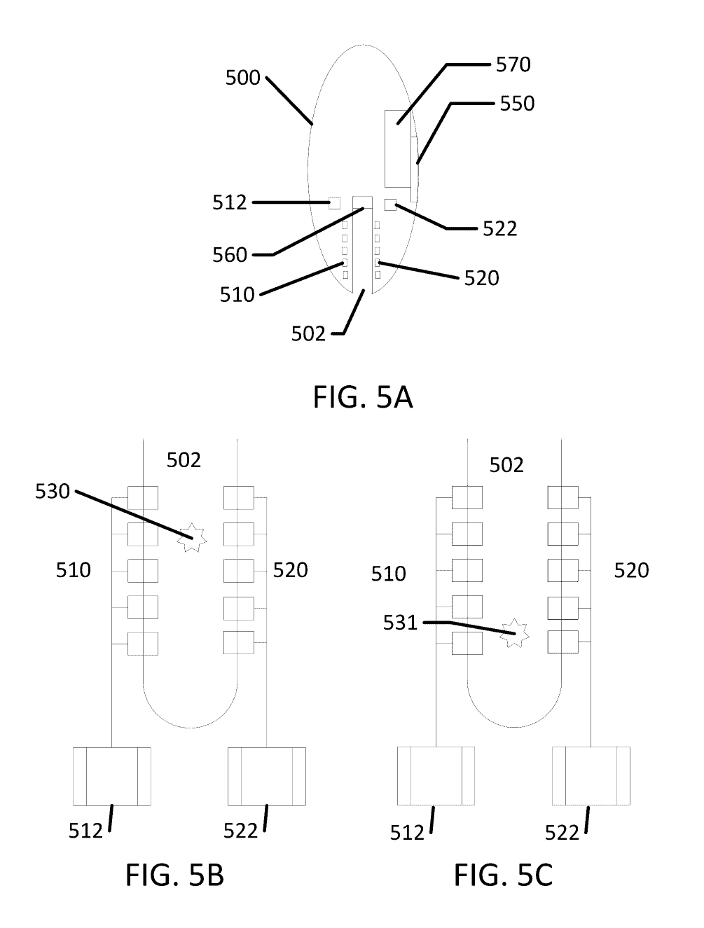

Once excited by a current (such as from the power management unit 420), the quantum-dot layer may emit light at a designed specified wavelength from the quantum-dot spectrometer system 410. The emitted light may interact with an outside environment, or with a specific sample or samples in the environment, wherein the sample or samples may absorb the emitted light at certain wavelengths. The quantum-dot spectrometer may then receive the remaining light, which has been transmitted through the sample or samples, in a quantum-dot detector (refer to FIG. 5A for one exemplary embodiment) within the quantum-dot spectrometer system 410.

Similarly, millimeter or nanometer sized quantum-dot detectors may be implemented into a quantum-dot spectrometer system 410. Current quantum-dot detectors may rely on charged-coupled devices (CCD); however, CCDs do not currently provide the size scale required for millimeter- or nanometer sized quantum-dot spectrometers. Rather, smaller photodiode arrays may be used to achieve the size requirements. Photodiodes are semiconductor devices that convert light into energy. Millimeter or nanometer sized photodiodes may be constructed through photolithographic means.

### Biomarkers/Analytical Chemistry

A biomarker, or biological marker, generally refers to a measurable indicator of some biological state or condition. The term is also occasionally used to refer to a substance the presence of which indicates the existence of a living organism. Further, life forms are known to shed unique chemicals, including DNA, into the environment as evidence of their presence in a particular location. Biomarkers are often measured and evaluated to examine normal biological processes pathogenic processes, or pharmacologic responses to a therapeutic intervention. In their totality, these biomarkers may reveal vast amounts of information important to the prevention and treatment of disease and the maintenance of health and wellness.

Biomedical devices configured to analyze biomarkers may be utilized to quickly and accurately reveal one's normal body functioning and assess whether that person is maintaining a healthy lifestyle or whether a change may be required to avoid illness or disease. Biomedical devices may be configured to read and analyze proteins, bacteria, viruses, changes in temperature, changes in pH, metabolites, electrolytes, and other such analytes used in diagnostic medicine and analytical chemistry.

Biomedical Devices with Quantum-Dot Spectrometers

FIG. 5A illustrates an exemplary QD spectrometer system in a generic biomedical device 500. There may be numerous exemplary types of use environments in which the device illustrated in FIG. 5A may be used. In some examples, the generic device may be located in a body environment that contains fluids such as within the vascular system, within subdermal spaces or in other body environments with fluids and analytes within the fluids. The generic example may utilize a passive approach to collecting samples wherein a sample fluid passively enters a channel 502 The channel 502 may be internal to the biomedical device in some examples and in other examples, as illustrated; the biomedical device may surround an external region with a reentrant cavity which may have access to the fluid of the subdermal location. In some examples where the biomedical device creates a channel of fluid external to itself, the device may also contain a pore 560 to emit reagents or dyes to interact with the external fluid in the channel region. The biomedical device 500 may contain regions that emit medicament 550 as well as regions that analyze surrounding fluid for the presence of an analyte, where the analyte may be the medicament for example. The device may contain controller 570 regions proximate to the medicament where control of the release of the medicament may be made by portions of the biomedical pill device. An analysis region may comprise a reentrant channel within the generic device that allows external fluid to passively flow in and out of the channel. When an analyte, for example, diffuses or flows into the channel it becomes located between the analysis region as depicted in FIG. 5A. The generic device may comprise numerous other functional regions such as a QD emitter controller 512, a QD sensor controller 522, a QD emitter 510 and QD receivers 520 as non-limiting examples

Referring now to FIG. 5B, once an analyte diffuses or otherwise enters the quantum-dot spectrometer channel which shall be referred to as the channel 502, a sample 530 may pass in the emission portion of a quantum-dot (QD) emitter 510. The QD emitters 510 may receive information from a QD emitter controller 512 instructing the QD emitters 510 to emit an output spectrum of light across the channel 502.

In some examples, the QD emitter may act based on emission properties of the quantum-dots. In other examples, the QD emitter may act based on the absorption properties of the quantum-dots. In the examples utilizing the emission properties of the quantum-dots, these emissions may be photostimulated or electrically stimulated. In some examples of photostimulation, energetic light in the violet to ultraviolet range may be emitted by a light source and absorbed in the quantum-dots. The excitation in the QD may relax by emitting photons of characteristic energies in a narrow band. As mentioned previously, the QDs may be engineered for the emission to occur at selected frequencies of interest. In a similar set of examples, QDs may be formed into the layered sandwiched mentioned previously between electrically active layers that may donate electrons and holes into the QDs. These excitations may similarly emit characteristic photons of selected frequency. The QD emitter 510 may be formed by inclusion of nanoscopic crystals, that function as the quantum-dots, where the crystals may be controlled in their growth and material that are used to form them before they are included upon the emitter element.

In an alternative set of examples, where the QDs act in an absorption mode, a combination of a set of filters may be used to determine a spectral response in a region. This mechanism is described in a prior section in reference to FIG. 2. Combinations of QD absorption elements may be used in analysis to select regions of the spectrum for analysis.

In either of these types of emission examples, a spectrum of light frequencies may be emitted by QD emitter 510 and may pass thru the sample 530. The sample 530 may absorb light from some of the emitted frequencies if a chemical constituent within the sample is capable of absorbing these frequencies. The remaining frequencies that are not absorbed may continue on to the detector element, where QD receivers 520 may absorb the photons and convert them to electrical signals. These electrical signals may be converted to digital information by a QD sensor controller 522. In some examples the QD sensor controller 522 may be connected to each of the QD receivers 520, or in other examples the electrical signals may be routed to centralized electrical circuits for the sensing. The digital data may be used in analyzing the sample 530 based on pre-determined values for QD wavelength absorbance values.

In FIG. 5C, the QD system is depicted in a manner where the sample is passed in front of spectral analysis elements that are spatially located. This may be accomplished for example in the manners described for the microfluidic progression. In other examples, the sample 530 may contain analytes that diffuse inside a region of a biomedical device that encloses external fluid with material of the biomedical device to form a pore or cavity into which the sample may passively flow or diffuse to an analytical region that passes light from emitters within the biomedical device, outside the biomedical device, and again to detectors within the biomedical device. FIGS. 5B and 5C depict such movement as the difference between the locations of the sample 530 which has moved along the analysis region to the new location 531. In other examples the QDs may be consolidated to act in a single multidot location where the excitation means and the sensing means are consolidated into single elements for each function. Some biomedical devices such as quantum dot devices may have space limitations that don't allow for a spectrometer comprising more than a hundred quantum-dot devices, but other biomedical devices may have hundreds of quantum-dot devices which allow for a full spectrographic characterization of analyte containing mixtures.

The QD analytical system may also function with microfluidic devices to react samples containing analytes with reagents containing dyes. The dye molecules may react with specific analytes. An example of such a binding may be with Förster resonance energy transfer (FRET) indicators. The dye molecules may have absorption bands in the ultraviolet and visible spectrum that are significantly strong, which may also be referred to as having high extinction coefficients. Therefore, small amounts of a particular analyte may be selectively bound to molecules that absorb significantly at a spectral frequency, which may be focused on by the QD analytical system. The enhanced signal of the dye complex may allow for more precise quantification of analyte concentration.

In some examples, a microfluidic processing system may mix an analyte sample with a reagent comprising a dye that will bind to a target analyte. The microfluidic processing system may mix the two samples together for a period that would ensure sufficient complexing between the dye and the analyte. Thereafter, in some examples, the microfluidic processing system may move the mixed liquid sample to a location containing a surface that may bind to any uncomplexed dye molecules. When the microfluidic system then further moves the sample mixture into an analysis region, the remaining dye molecules will be correlatable to the concentration of the analyte in the sample. The mixture may be moved in front of either quantum-dot emission light sources or quantum-dot absorption filters in the manners described.

A type of fluorescent dye may be formed by complexing quantum-dots with quenching molecules. A reagent mixture of quantum-dots with complexed quenching molecules may be introduced into a sample containing analytes, for example, in a microfluidic cell, within a biomedical device. The quenching molecules may contain regions that may bind to analytes selectively and in so doing may separate the quenching molecule from the quantum-dot. The uncomplexed quantum-dot may now fluoresce in the presence of excitation radiation. In some examples, combinations of quantum-dot filters may be used to create an ability to detect the presence of enhanced emission at wavelengths characteristic of the uncomplexed quantum-dot. In other examples, other manners of detecting the enhanced emission of the uncomplexed quantum-dots may be utilized. A solution of complexed quantum-dots may be stored within a microfluidic processing cell of a biomedical device and may be used to detect the presence of analytes from a user in samples that are introduced into the biomedical device.

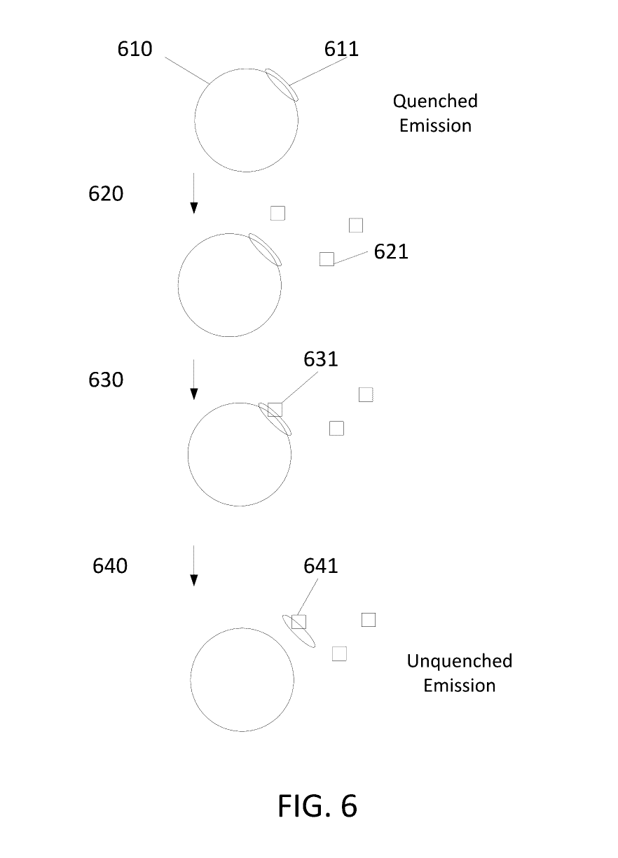

Referring to FIG. 6, an exemplary illustration of the concept of complexed quantum-dots acting as a dye is illustrated. A quantum-dot 610 may comprise an exemplary material such as indium phosphide/zinc sulfide, copper indium sulfide/zinc sulfide, cadmium selenide, cadmium sulfide, lead sulfide, lead selenide, indium arsenide, and indium phosphide as examples. Other examples may comprise nanoparticles of silicon and carbon. Any material that may form a strained band structure of the type characteristic of a quantum-dot may be used in various embodiments. The quantum-dot core may be surrounded by a core shell coating that provides an interface from the quantum-dot to its outside environment. For some examples, a biocompatible lipid coating which may allow for the binding of quenching molecules to the surface of the dot may also be provided. The quenching molecules 611 may be bound to the quantum-dot surface and may act to facilitate electronic energy transfer from the quantum-dot which may result in a deexitation of the quantum-dot energy without fluorescent emission. A solution of the quantum-dots may be mixed 620 with a sample containing analytes 621. During the mixing, analytes may complex 630 with the quenching molecules forming an analyte/quenching molecule complex 631. The complexing of the analyte with the quenching molecule may decouple 640 the quenching molecule from the quantum-dot, resulting in a free analyte/quenching molecule 641 and an uncomplexed quantum-dot. Now, the quantum-dot may be excited by photons at energy distinct from the inherent fluorescent energy of the quantum-dot and the unquenched quantum-dot will now fluoresce. The concentration of the analyte in the sample may be a function of the fluorescence signal emanating from the unquenched quantum-dot. An electroactive biomedical analysis system may include a light source, which may be a quantum-dot light emitting diode for example or other light sources with energy distinct from the fluorescence signal. A detector may be configured to detect all light that traverses a spectral analysis region. Alternatively, quantum-dot absorbance filters or other light filters may be used to selectively pass the energy band of the quantum-dot fluorescence signal.

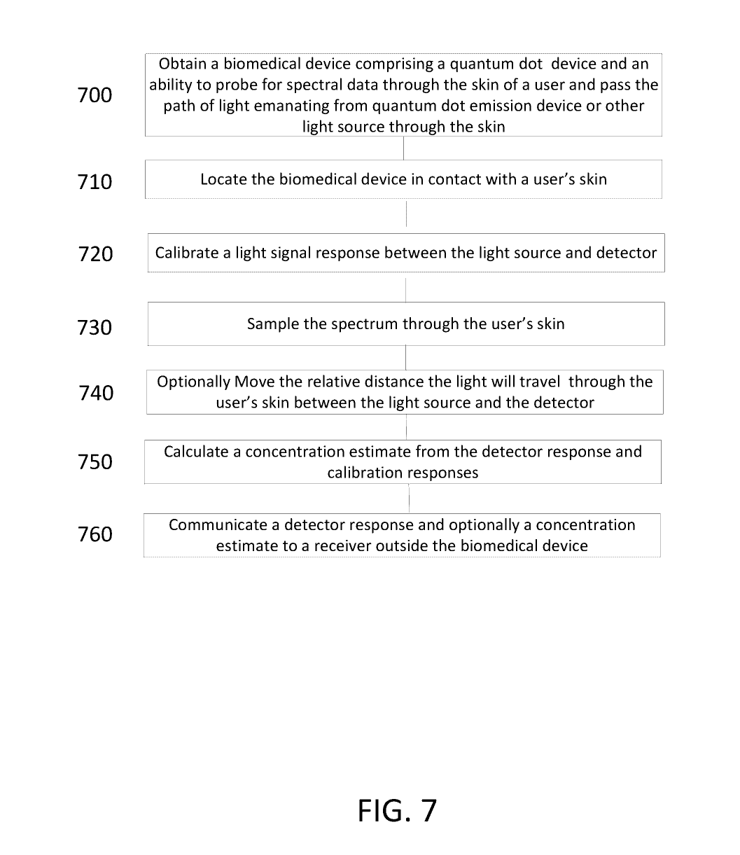

Referring to FIG. 7, an illustration of a flow diagram for analyte analysis in quantum-dot configured biomedical devices is provided. At step 700, a user may obtain a biomedical device comprising: a quantum dot device and an ability to probe for spectral data through the skin of a user and pass the path of light emanating from quantum dot emission device or other light source through the skin. The light source may include a quantum-dot light emitting diode or a set of quenched quantum-dot filters configured to isolate selected spectral regions for analysis. Other light sources such as light emitting diodes and lasers may also be used. In some examples, the biomedical device may be obtained by an intermediary for the purposes of use by an end user. It is important to note that the biomedical device may comprise any suitable device configured for measuring a particular material. At step 710, the biomedical device may be located in contact with a user's skin. The location may include regions proximate to thin flaps of skin such as, in non-limiting examples, ear lobes, finger web spaces or other thin probe compatible flaps of skin. Or, the location may include flat skin locations with underlying flexibility, such as the region of the vastus lateralis on the top of a user's leg, the region around the pectoral muscles. and the region around the biceps muscles as non-limiting examples.

At step 720, the biomedical device may be used to generate a calibration response of the light sources and detectors. In some examples, the biomedical device may be packaged with a simulant between the light source and detector, or a calibration standard may be used to sample the spectrum through the simulant or calibration sample.

At step 730, the biomedical device may be attached to the user's skin region under study and a sample of the spectrum may be obtained. In some examples, the biomedical device may be in the form of an adhesive patch which attaches to the skin. Although the biomedical device may be calibrated as mentioned in step 720, there may be manners of calibrating the signal obtained from spectral analysis by varying factors of the sampling such as the thickness of the tissue that light travels through. At step 740, this variation in the thickness of skin that is sampled may be performed in some examples.

At step 750, in some examples, the biomedical device may include on-board processing devices and software algorithms that may allow for a calculation of an estimate of a concentration of the analyte in the sample. In other examples, the raw data signals, detector (calibration) signals, and detector signals through the skin may be transmitted without further signal processing in the biomedical device. At step 760 the raw data signals may be communicated, for example, via wireless communication, to an external transceiver. In some examples, a calculated estimate of the concentration of an analyte may also be communicated. In still further examples, there may be numerous other sensor data that may be transmitted in addition to the analysis system data, which may include in a non-limiting perspective sensor measurements of the temperature sensed in a region of the biomedical device.

The QD spectrometer systems may be utilized in several different biomedical devices including: clips to grab flaps of skin, wristlets and cuffs that surround tissue, bandages which attach to skin and allow the probing of the tissue in the skin. The information obtained from the QD spectrometer system may be utilized for biometric analysis such as real-time readings of glucose for diabetics, as a non-limiting example. The information obtained may be communicated to a tertiary device, such as a smart phone.

### Methods for Monitoring Bioanalytes

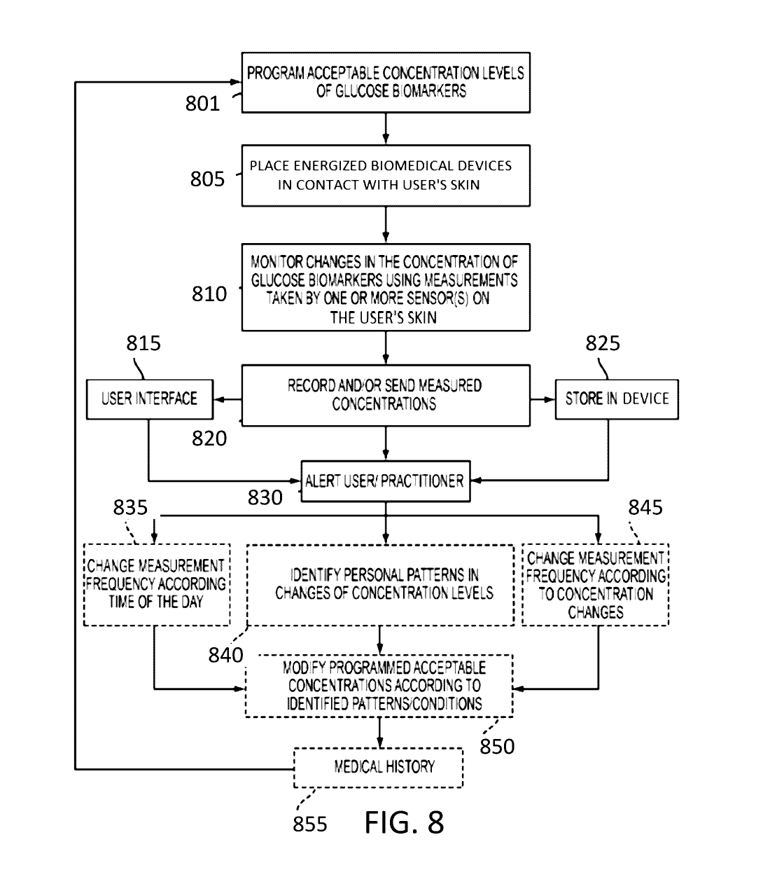

Referring now to FIG. 8, exemplary method steps that may be used to monitor analyte levels of a user wearing a non-invasive quantum dot spectrometer according to aspects of the present invention are illustrated. At step 801, thresholds values may be programmed into a software program. According to aspects of the present invention, threshold values may include, for example, acceptable levels for the concentration of glucose biomarkers determined by non-invasive measurement through the skin with biomedical devices. In some examples, these biomedical devices may comprise quantum dot light sources or filters in their detection scheme. The measurement of levels of biomarkers may be used to monitor different conditions such as depression, high blood pressure, and the like, are also within the inventive scope of aspects of the present disclosure. In addition, the preprogrammed levels may be different depending how the light source travels through the skin. The program may be stored and executed using one or both a processor forming part of the biomedical device and an exterior or external device in communication with the processor. An exterior or external device may include a smart phone device, a PC, a specialized biomedical device user interface, and the like; and may be configured to include executable code useful to monitor properties of tissues proximate to the skin. Skin properties may be measured by one or more sensors contained in the biomedical device. Sensors may include electrochemical sensors and/or photometric sensors. In an exemplary embodiment, the sensor analysis step may relate to a photometric sensing of glucose concentration based on a QD spectrometry.

At step 805, the biomedical device including a quantum dot spectrometer system may be placed in contact with a portion of the user's skin surface and worn. At step 810, concentration changes of biomarkers may be monitored using the one or more sensors. The monitoring of the biomarkers may occur at a predetermined frequency/bandwidth or upon demand through a user interface and/or an activation sensor in the non-invasive quantum dot device. Biomarkers can include those correlated to glucose levels, depression, blood pressure and the like.

At step 820, the processor of the non-invasive quantum dot device can record the measured property/condition from passing light through the skin region. In some embodiments, the processor of the non-invasive device may store a record of the measured property and/or send it to one or more device(s) in communication with the non-invasive quantum dot device. At step 815, the value recorded can be stored and analyzed in the user interface in communication with the non-invasive quantum dot device, and/or, at step 825, the analysis and recording can take place in the non-invasive quantum dot device.

At step 830, one or both the non-invasive quantum dot device and the user interface may alert the user, and/or a practitioner, of the measured concentration. The alert may be programmed to occur when the levels measured are outside the predetermined threshold values programmed, received and/or calculated by the non-invasive quantum dot device. In addition, in some embodiments, the data and alerts may be analyzed to perform one or more steps of: a) change measurement frequency according to the time of the day, b) identify personal patterns in the changes of concentration levels measures, and c) change the measurement frequency according to the changes in concentrations measured.

At step 835, the time of the day may change the frequency of measurements. For example, if the non-invasive quantum dot device is one that would remain on the skin during sleep, the number of measurements during 10 PM and 6 AM can decrease or stop. Similarly, during lunch and dinner times the frequency may increase to detect changes due to the food consumption of the user. At step 840, patterns in changes of the concentration levels may be identified by the system. Using the identified patterns, the system may alert the user of causes and/or, at step 845, change the frequency according to the identified changes so that the system is more alert during critical identified conditions. Critical conditions can include events that would trigger a significant increase or decrease in glucose levels. Events can include, for example, holiday dates, exercise, location, time of the day, consumption of medicaments and the like.

In some embodiments, at step 850, the originally programmed values may be customized, periodically or in real time, according to identified patterns/conditions. This ability may allow the system to increase its effectiveness by eliminating false alarms and increasing sensitivity at a critical condition. Effectiveness can promote user participation with the system thereby maximizing the benefits of the non-invasive quantum dot device and thereby providing a safe monitoring system. At step 855, data relating to the user including, for example, the identified patterns, measurements, and/or preferences may become part of the medical history of the user. Medical history may be stored securely by encrypting the data and/or restricting its access.

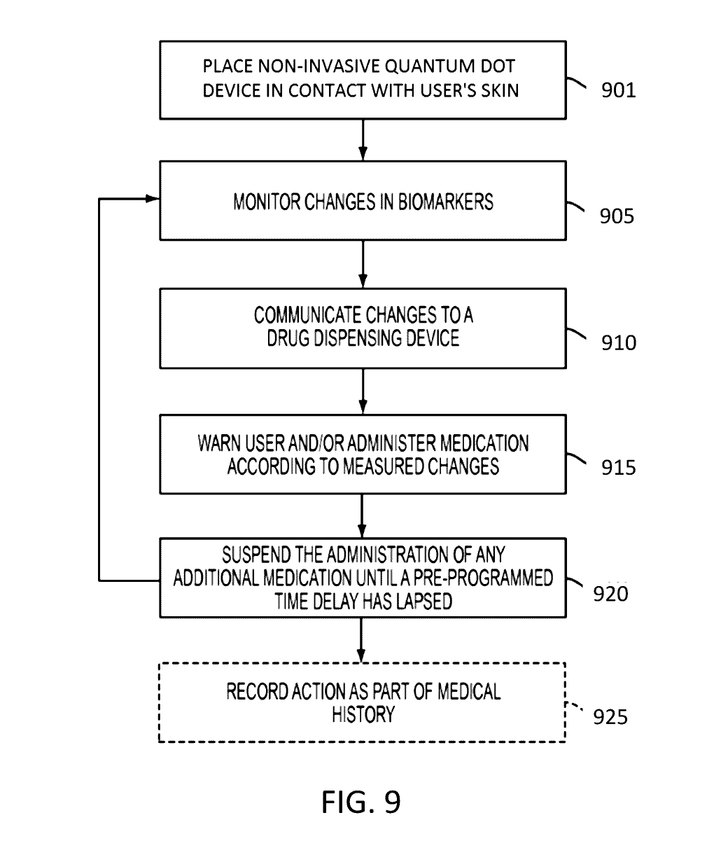

Referring now to FIG. 9, exemplary method steps that may be used to treat the glucose levels of a user wearing the non-invasive quantum dot device according to aspects of the present invention are illustrated. At step 901, a non-invasive quantum dot device including a QD spectrometer analytical system is placed in contact with a user's skin. In other embodiments, the non-invasive quantum dot device may be, for example, in the form of an intraocular device or a punctal plug, and still include aspects of the QD spectrometer system described in the present disclosure.

At step 905, changes in biomarkers in the ocular fluid can be monitored as in the case of a contact lens. Methods of monitoring the biomarker changes may include, for example, steps illustrated in FIG. 8. At step 910, measured changes can be communicated in real time to a medicament-dispensing device in direct or indirect communication with the non-invasive quantum dot device. Although the changes in concentration of the monitored biomarkers in ocular fluid may include a time delay in relation to the concentration changes in the bloodstream of the user, upon detection, at step 915 the medicament-dispensing device may administer a medicament capable of lowering or raising concentrations to a normal level. For example, glucose levels may be monitored and treated when they are outside a normal level. Continuous monitoring may prevent uncontrolled blood sugar levels which may damage the vessels that supply blood to important organs, like the heart, kidneys, eyes, and nerves. Because an individual whose glucose levels may reach a level that exposes him/her to the risks may feel fine, aspects of the present disclosure may help take action upon early detection of the condition. Early detection may not only bring back levels to a normal condition and/or make the user aware, but additionally prevent the more dramatic and permanent consequences including, for example, a heart attack or stroke, kidney failure, and blindness which have been known to occur when abnormal glucose levels are left untreated.

In addition, in some embodiments the medicament administering device may send an alert to the user through its interface or using component of the non-invasive quantum dot device. For example, in some non-invasive quantum dot device embodiments the media insert may include a light projection system, such as one or more LEDs, capable of sending a signal to the user

Subsequently at step 920, any further drug administering can be suspended to prevent overdosing of the system due to the time delay of the effect of the drug and the effect to be reflected in the tear fluid. For example, the medicament may require 10-30 minutes to counteract the abnormal level, and upon its effect, may take another 20 minutes to equalize concentrations in tear fluid. Consequently, programmed algorithms capable of correlating the condition, time delay, and appropriate subsequent dosing of medicaments can be programmed in the system to function safely. At step 925, data relating to one or both the measured conditions and the medicament administration to the user may be stored and used as part of a treatment and/or medical history of the user.

As mentioned previously, the construct of a media insert, which has frequently been described in reference to ophthalmic examples where a hydrogel skirt surrounds the media insert, may have particular relevance in various exemplary types of quantum dot spectrometer devices. The media insert may comprise internal components that allow for a QD spectrometer to function in a small form factor, with an energy source, the light sources, the detectors, quantum dot filters and/or emitters and other components to measure and transmit spectral responses. The media insert may significantly encapsulate these various components to ensure that fluids and tissues in the vicinity of the media insert are not exposed to chemicals of the various components. These various media insert examples may be themselves surrounded in various coating layers which may include hydrogel layers as well. In some other examples, the media insert may create an efficient manner to organize the various components of a quantum dot spectrometer even when the QD spectrometer is located externally to a user's skin layers by supporting acquisition of spectra through the skin layers.

### Non-invasive Monitoring Through a User's Skin

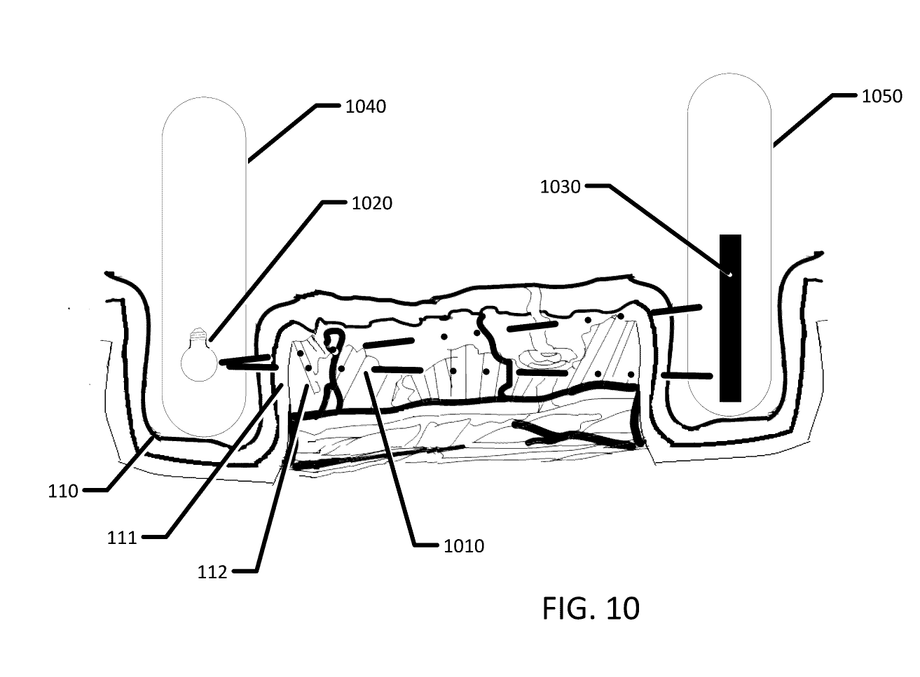

Referring to FIG. 10, a cross section of a layer of skin with exemplary quantum dot monitoring devices is illustrated. In some examples, a light source 1020 and a detector 1030 may be impressed into a user's skin so that the path of light may run through subcutaneous layers and then back out to the detector. In the example, a casing 1040 and 1050 around a light source 1020 or a detector 1030 is depressed into the skin of a user. The epidermal layer 110 may be depressed as well as the dermis layer 111 and the subdermal layers 112. Light may emerge from the light source 1020 and proceed through the fluid and tissue layers 1010 before emerging at the detector 1030 which may also be depressed into the skin to allow for the detectors to intercept the light. In some examples, quantum dot structures may filter the light that emerges into the skin as shown by the dotted lines. In other examples, the light source 1020 may excite the quantum dots to cause them to emit light. In other examples, quantum dot structure may be located at the detector and filter the light before it is detected.

The detection of analytes may benefit by an abundance of sample data that may be used to investigate small signals that may be imbedded in a large amount of background data and noise. It may also be important to be able to vary parameters that may vary along with the signal. As well, with quantum dots, an ability to sample a spectrum from numerous quantum dots also improves the ability to uniquely identify analytes and the amounts of them. The means of sampling as described in FIG. 10, allow for varying the length of skin that is sampled with the absorption detectors. As well by increasing the pressure that the light source and the detector are pressed into the skin, the depth of probing of the skin may be varied.

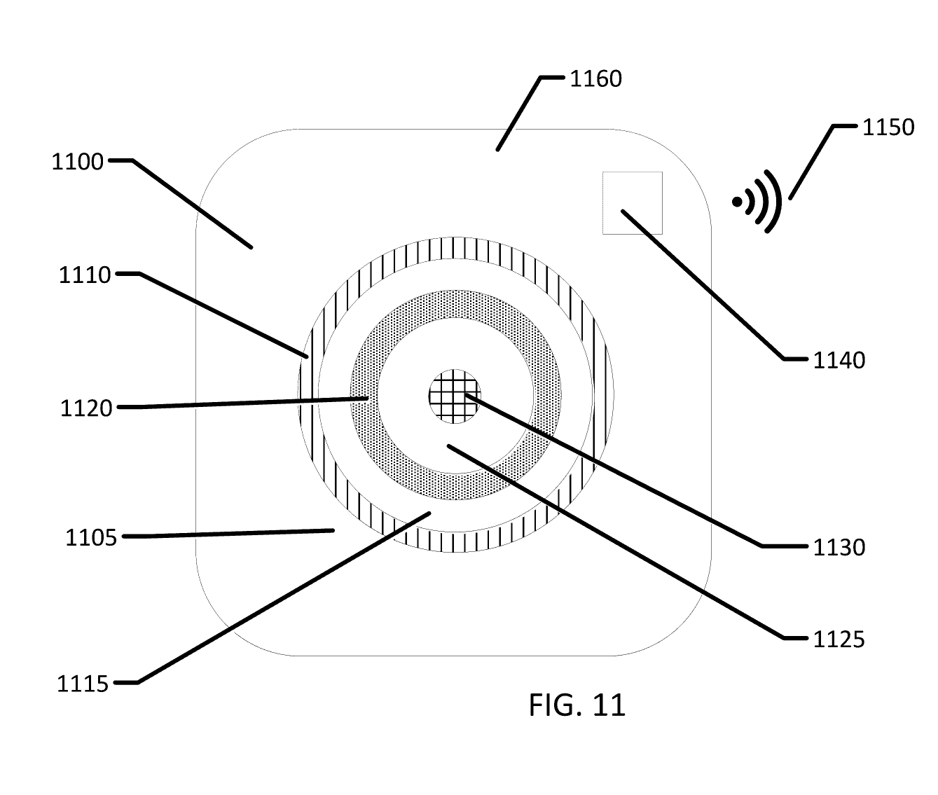

Referring to FIG. 11, an exemplary bandage device 1100 is illustrated which may be used to implement the measurement methods related to FIG. 10. Although the example includes a bandage, in some examples the device may include a cuff, strap, clip or other such attachment device. The bandage may have an adhesive layer 1105 that continues between layers of detectors at adhesive layers 1115 and 1125. The light source 1130 may be located at a central point. In some examples, detector rings 1110 and 1120 may be mounted on an electro-active expansion element. In some examples, the electro-active expansion elements may comprise piezoelectric elements, electroactive elastomeric elements or other elements that may expand on application of electrical potential. In a detector ring, the quantum dot elements may be arranged around the detector rings. In some examples the quantum dot elements may be located with gaps between them, such that light emanating from the light source 1130 may travel through the skin and intercept a detector in detector rings 1110 and 1120. The depth of the impression of the light source and detectors may be varied by application of electro-potential to the electro-active expansion elements. The exemplary bandage device may include electronics 1140 and energization elements 1160 such as a battery included into the body of the bandage. The electronics 1140 may control the operation of the expansion elements under the detector rings 1120 and 1110 and the light source 1130. As well, the electronics may be able to communicate 1150, in some examples wirelessly, with external devices to pass on control information as well as to communicate the detected data from the quantum dot analysis system.

The data may be passed onto to servers with large data calculating capability. Large amounts of collected data may be processed to extract signal related to desired analyte quantification. In some examples, the full spectral capabilities of the quantum dot analysis system may be able to extract multiple different analyte signatures from the analysis of light absorption of the user's tissue. In some examples, the large data calculating capability may include hardware and software capable of processing the data using cognitive computing where patterns may be extracted from various data streams that may improve the accuracy of calculations relating to a particular desired analyte. For example, combinations of data from many different users may allow for the recognition of common patterns for how glucose varies during a day and during events such as meals and exercise events. Combination of other biometric sensors along with the quantum dot spectroscopy devices may allow for large data analysis systems equipped with cognitive computing capabilities to recognize the time variable trend of a signal extracted from the spectral data. So, even if there are competing analytes that may confuse the accuracy of an analysis, the time dependent and event dependent trends of the data may represent patterns that may be cognitively recognized and used to enhance the accuracy of the data.

The radial nature of the example of FIG. 11 allows for the simultaneous variation of path length in the tissue of the user. The path length variation may allow for extractions of effects that occur on the top layer of the skin such as the epidermis from the bulk effect. The technique of compressing the light source and the detectors into the skin naturally produces a portion of the light path that proceeds through the skin in two passes. The other portion of the light path is what is varied by spacing the detectors out along two different radial paths with different radii. Since both radial dimensions with pass through the surface layers twice, effects relating to skin coloration and other aspects of the surface of the skin, the effect of these surface layers may be extracted from other spectral effects of analytes in beneath the skin. Therefore, by varying the path from the central light source to the first radius and then to the second radius on a routine basis the difference between the two signals is related to the different path length and not due to the surface layers of the skin or coloration of those layers.

Specific examples have been described to illustrate sample embodiments for the methods and devices related to inclusion of quantum-dots for spectroscopic analysis in biomedical devices. These examples are for said illustration and are not intended to limit the scope of the claims in any manner. Accordingly, the description is intended to embrace all examples that may be apparent to those skilled in the art.

What is claimed is:

1. A biomedical device comprising:
an energization element;
a quantum-dot spectrometer including a quantum-dot light emitter, a photodetector, and a means of communicating information from the quantum-dot spectrometer to a user, wherein the quantum-dot spectrometer is powered by the energization element; and
a bandage device, wherein the bandage device contains the energization element and the quantum-dot spectrometer, and wherein the bandage device includes an electroactive elastomeric element that presses the quantum-dot light emitter and the photodetector to depress skin of the user.

2. A biomedical device comprising:
an energization element including a first and second current collector, a cathode, an anode, and an electrolyte;
a quantum-dot spectrometer including a light emitter, a quantum-dot photodetector, and a means of communicating information from the quantum-dot spectrometer to a user, wherein the quantum-dot spectrometer is powered by the energization element; and
a bandage device, wherein the bandage device contains the energization element and the quantum-dot spectrometer, and wherein the bandage device includes an electroactive elastomeric element that presses the quantum-dot photodetector and the light emitter to depress skin of the user.

3. The biomedical device of claim 2 wherein the quantum-dot photodetector comprises a first component and a second component deployed at a different length from the light emitter.

4. The biomedical device of claim 3 wherein the first component is a circular device of a first radius and the second component is a circular device of a second radius.

5. The biomedical device of claim 4 wherein the quantum dots are located along the first component and the second component.

6. The biomedical device of claim 5 wherein quantum dots are located upon solid portions of the first component with gaps between consecutive solid portions, wherein the gaps allow light from the light emitter to continue through the skin and on to the second component.

7. The biomedical device of claim 6 further comprising a wireless communication device.

8. The biomedical device of claim 7 wherein the wireless communication device communicates data which is received at a server comprising algorithms to analyze the data for presence of analytes.

9. The biomedical device of claim 7 wherein the server comprises a cognitive computing function.

10. A biomedical device comprising:
an energization element including a first and second current collector, a cathode, an anode, and an electrolyte;
a quantum-dot spectrometer including a quantum-dot light emitter, a photodetector, and a means of communicating information from the quantum-dot spectrometer to a user, wherein the quantum-dot spectrometer is powered by the energization element; and
a cuff device, wherein the cuff device contains the energization element and the quantum-dot spectrometer, and wherein the cuff device includes an electroactive elastomeric element that presses the quantum-dot light emitter and the photodetector to depress skin of the user.

11. The biomedical device of claim 10 wherein the photodetector comprises a first component and a second component deployed at a different length from the quantum-dot light emitter.

12. The biomedical device of claim 11 wherein the first component is a circular device of a first radius and the second component is a circular device of a second radius.

13. The biomedical device of claim 12 wherein the quantum dots are located along the first component and the second component.

14. The biomedical device of claim 13 wherein quantum dots are located upon solid portions of the first component with gaps between consecutive solid portions, wherein the gaps allow light from quantum-dot light emitter to continue through the skin and on to the second component.

15. A method of analyzing analytes comprising:
fabricating a quantum-dot photodetector onto a biomedical device;
fabricating a photon emitter onto the biomedical device;
connecting the quantum-dot photodetector and light emitter to an integrated circuit controller within the biomedical device wherein the integrated circuit controller is capable of directing a functionality of the quantum-dot photodetector and emitter;
emitting a wavelength band from the photon emitter;
receiving transmitted photons into the quantum dot photodetector; and
analyzing an absorbance of an analyte based on an intensity of photons received;
wherein the biomedical device comprises an energization element including a first and second current collector, a cathode, an anode, and an electrolyte; and
wherein the quantum-dot photodetector is powered by the energization element.

16. The method of claim 15 further comprising connecting an electroactive elastomeric element to the quantum dot photodetector and the light emitter, wherein an electrical signal applied to the electroactive elastomeric element depresses the quantum dot photodetector and the light emitter into skin of a user.

17. A method of analyzing analytes comprising:
obtaining a biomedical device comprising a quantum dot, wherein the biomedical device comprises a photon source and a first photodetector which provide an ability to probe for spectral data through skin of a user and pass light through the skin of the user, wherein a distance between the photon source and the first photodetector is a first distance;
locating the biomedical device in contact with the user's skin;
measuring absorption through the user's skin with the photon source and the first photodetector;
locating a second photodetector with a second distance between the photon source and the second photodetector;
measuring absorption through the user's skin with the photon source and the second photodetector; and
communicating the data from the biomedical device to an external receiver.

18. The method of claim 17 wherein the first photodetector comprises quantum dots.

19. A method of monitoring a patient's glucose level comprising:
associating a data level in a controller, wherein the data level corresponds to acceptable detection measurements obtained with a biomedical device comprising a quantum dot, wherein the detection measurements correlate to a concentration of the glucose;
placing the biomedical device comprising a quantum dot in contact with a patient's skin, wherein the biomedical device comprises a photon source and at least a first photodetector which provide an ability to probe for spectral data through skin of the patient;
monitoring the detection measurements which correlate to the concentration of the glucose in the patient's body, wherein the detection measurements are obtained non-invasively through the skin of the patient;
communicating the detection measurements to at least one of the patient and a medical practitioner of the patient;
identifying a pattern in the detection measurements that are tied to the patient; and
adjusting the data levels in the controller based on the pattern.

20. A method of administering a medication comprising:
placing a biomedical device comprising a quantum dot in contact with a patient's skin, wherein the biomedical device comprises a photon source and at least a first photodetector which provide an ability to probe for spectral data through skin of the patient, wherein the first photodetector comprises quantum dots;
monitoring detection measurements which correlate to a concentration of glucose in a patient's body, wherein the detection measurements are obtained non-invasively through the skin of the patient;
communicating the detection measurements to a drug dispensing device;
administering the medication in response to the communicated detection measurements;
suspending administration of additional medication until either of an elapsing of a predefined time period or an override signal communication from a practitioner of the patient; and
recording the detection measurement and administration event details in a database.
